(19) United States
(12) Patent Application Publication
(10) Pub. No.: US 2008/0167517 A1
(43) Pub. Date: Jul. 10, 2008

(54) Prasanti-loka (bliss in mind) & method to teach

(76) Inventor: Maheswar R. Mikkilineni, Washington, DC (US)

Correspondence Address: M.R. MIKKILINENI, 1510, 44th St. NW, WASHINGTON, DC, 20007, US

(21) Appl. No.: 11/999,349

(22) Filed: Dec. 5, 2007

Related U.S. Application Data
(63) Continuation-in-part of application No. 11/449,519, filed on Jun. 8, 2006, now abandoned.

(51) Int. Cl. A61M 21/00

(52) U.S. Cl. 600/26

(57) ABSTRACT

Method to create bliss in mind and induce sleep includes steps in its first preferred embodiment: 1. Calming mind of intrusive thoughts or decreasing of excited-signals in the brain to prepare for a sleep cycle by concentrating upon one principal thought. 2. Splitting mind to activate master-clock in the brain to induce a sleep cycle by concentrating upon two or more principal thoughts. Method to teach for sleep includes steps in its second preferred embodiment: 1. Identify reason(s) for intrusive thoughts and select appropriate principal thoughts. 2. Practice and learn to calm mind using one principal thought. 3. Practice and learn to split mind and fall-asleep using two or more principal thoughts at the same time.

### BACKGROUND OF THE INVENTION

1. Field of the Invention

The present invention is a process to create peace in mind or induce sleep, and a method to achieve peace by learning to use the process-algorithm and transform his/her brain-neurons into a different-physical state to produce melatonin (dopamine) and serotonin on its own, without the use of drugs or devices. The development work is in progress to build a ‘device’ that can assist a person requiring more help to practice and learn the skill of the art or the method to achieve the desired result.

2. Background Information

The brain is the control center of the body. It controls thoughts in the mind, signals-senses, memory and the function of organs-cells. Survival depends on a properly functioning brain to regulate the functions of the organs, so that the heart pumps blood, the lungs breath oxygen-air, and the digestive system assimilates water-food. Brain requires a sleep-cycle, deep sleep (DS) and rapid eye movement (REM) sleep, of about 8-hours in a 24-hour day to function properly. A recent study (see REF.-2) at Harvard Medical School and University of California concludes—

“ . . . a lack of sleep causes the brain's emotional centers to dramatically overreact . . . (with) psychiatric disorders . . . (and) fractures the brain mechanisms that regulate key aspects of mental health . . . and, sleep appears to restore emotional brain's circuits . . . ”

Thus, sleep-problems play a key-role in a large number of brain disorders. For example, strokes and asthma attacks tend to occur more frequently due to changes in hormones, heart rate, breathing rate, and other changes associated with sleep. Sleep deprivation triggers seizures in some types of epilepsy. Neurons that control sleep interacts closely with the immune system—sleep may help body conserve energy that the immune system needs to mount an attack. Sleep problems occur in almost all people with mental disorders, including those with depression and schizophrenia. A person with depression is often awake in the night and find unable to get back to sleep. Extreme sleep deprivation can lead to a seemingly psychotic state of paranoia and hallucinations in otherwise a healthy person, and disrupted sleep can trigger episodes of mania—agitation and hyperactivity, to a person with manic depression.

The National Sleep Foundation's (NSF) sleep in America poll found that 74% of American adults experience sleep problem a few nights a week or more, 39% get less than 7-hours of sleep each weeknight, and more than 1 in 3 (37%) are so sleepy during the day that it interferes with daily activities. An article ‘Counting sheep no aid to insomnia’ (see REF.-5) published in January 2002, on the findings of Oxford study on sleep, reported that—

“1 in 10 suffer from chronic insomnia and it is estimated that sleeplessness costs the US economy $35 billions a year . . . ”

The sleep problems arise from changes in the brain regions and neurons that control sleep, or from the drugs used to control other disorders. Once sleep problem develops, it can add to a person's impairment and cause confusion, frustration, or depression. Sleep-problems are common in many other disorders as well, including Alzheimer's disease, stroke, brain injury and cancer. In the December 2007 issue of The Lancet Oncology, the International Agency for Research on Cancer, the cancer arm of the World Health Organization, publishes the results of its analysis of an expert panel, and reports—

“Scientists suspect that shift work (night-work) is dangerous because it disrupts the circadian rhythm, the body's biological clock. The hormone melatonin, which can suppress tumor development, is normally produced at night. Light shuts down melatonin production, so people working in artificial light at night may have lower melatonin levels, which scientists think can raise their chances of developing cancer. Sleep deprivation may also be a factor. People who work at night are not usually able to completely reverse their day and night cycles. Not getting enough sleep makes your immune system vulnerable to attack, and less able to fight off potentially cancerous cells. Certain processes like cell division and DNA repair happen at regular times. But if the body needs to do something at an unusual time—like produce insulin in the middle of the night to help digest food—that can set off a chain reaction of biological mistakes. The problem is re-setting your body's clock. Anyone whose light and dark schedule was frequently disrupted—including frequent long-haul travelers or insomniacs—could face the same increased cancer risks. The balance between light and dark is very important for your body, and make sure to sleep in a darkened room—Just get a dark night's sleep.”

Circadian rhythms are regular changes in mental and physical characteristics that occur in the course of a day (circadian is Latin for “around a day”). Most circadian rhythms are controlled by the body's biological “master-clock” in the brain. The pattern of waking during the day when it is light and sleeping at night when it is dark is a natural part of life's circadian rhythm. Only recently have scientists begun to understand sleep alternating and how it is related to daylight and darkness. A key factor that regulates human sleep is the exposure to light or to darkness. Exposed light reaches photoreceptors in the retina of eye, creates signals to stimulate a nerve pathway which travel along the optic nerve from retina to an area in the brain called ‘thalamus-hypothalamus’. There, a special center called the supra-chiasmatic nucleus (SCN) initiates signals to other parts of the brain including sending excited-signals into the cortex which keeps a person wide awake. This SCN is called the “master-clock”, and it is actually a pinhead-sized brain structure that contains about 20,000 neurons, and rest just above the point where the optic nerves cross.

The SCN works to set a regulated pattern of sleep-cycle. Once exposed to the first light each day, the clock begins performing functions like raising body-temperature, rate of heat-beat and breathing, and releases stimulating hormones like cortisol. The SCN delays the release of other hormones like melatonin (dopamine), which is associated with sleep onset, until many hours later when darkness returns. The signals from SCN travel to several brain regions, including the pineal gland, which responds to light-induced signals by switching off production of the hormone melatonin. The body's level of melatonin normally increases after darkness falls, making a person feel drowsy. The SCN governs functions that are synchronized with the sleep-wake cycle, including body temperature, hormone secretion, urine production, and changes in blood pressure, heart rate and breathing rate etc. Because sunlight or other bright lights can reset the SCN, the biological cycles normally follow the 24-hour cycle of the sun. Disruptions in circadian rhythms increase the risk of heart problems, digestive disturbances, and emotional and mental problems, all of which may be related to sleep-problem. A blind person experiences life-long sleeping problem because his/her retina is unable to detect light. Such a person will have a kind of periodic insomnia because the circadian rhythms follow innate cycle rather than a 24-hour one. Daily supplements of melatonin may improve night-time sleep for such a person. However, since the high doses of melatonin found in most supplements can build up in the body and long-term use of this substance may create new problem.

Melatonin is a natural hormone made by the human body's pineal gland, which is a pea-sized gland located in the brain close to ‘thalamus’. During the day the pineal gland is inactive. When the sun goes down and darkness occurs, the pineal is supposed to be turned on by an active-SCN to begin producing melatonin and release into the blood. When SCN works, as it should, it results in inviting sleep to the brain. Melatonin levels in the blood stay elevated for about 12 hours, all through the night, before the light of a new day when it fall back to low daytime levels. Besides adjusting the timing of the body's clock, bright light has another effect—it directly inhibits the release of melatonin.

Currently, there are several prescription drugs available to aid sleep. They can shorten the time it takes to fall asleep and reduce awakenings, which adds to total time spent asleep. Possible side effects include feeling tired or drowsy the next day, memory loss, headache and problems with performance. Prescription sleeping pills can cause strange and potentially dangerous side effects. Those side effects can include dangerous allergic reactions and bizarre behaviors such as sleep eating, walking and driving, in which a person will drive a car while not fully awake and has no memory of doing so.

3. Description of the Prior Art

Reply to office action (Oct. 12, 2007)

@1—The drawings:
Reply—FIGS. 1, 2, 3.1-3.3 attached.

@2&4—The new matter:
Reply—The so-called ‘new matter’—the words and sentences cited by the Examiner is not a new matter; because, the process described in the original ‘possession of the claimed invention’ cannot change into a new matter as a result of adding more words to clarify the method at the request of prior-Examiner(s).

The original claim-description (see on p 9/11) consists of 2-parts: (1) Relax or empty-mind of (anxiety) and Split-mind calmly-equally upon two-spots, things or items at the same-time; and (2) Any person with ordinary skill can be trained to utilize the process to attain the desired result. The principal concept of the method can be summarized as—

‘concentrate upon principal thoughts to calm mind, and split the mind to turn-off the light switch in the brain-stem and activate master-clock to fall-asleep; and train person(s) to learn the skill of the art.’

That concept of the process cannot be changed, no matter how many words-sentences are added subsequently to clarify in making the Examiner see the difference between the instant invention and the ‘counting sheep’ or a game of mental-exercise for fun. The record shows, the prior-Examiner(s) asked for “a detailed description of the process . . . ” (see office action mailed on Feb. 27, 2007) which demanded for clarity of the “method for performing the process . . . steps in the process”. That made the inventor to separate the original claim into 2-claims: (1) Prasanti-loka (Bliss in mind) or Method to induce sleep, and (2) Method to Teach or Method to train . . . , and use more paragraphs with more words-sentences and biochemistry etc., in describing the process while keeping the original ‘possession of the claimed invention’ the same. The original process or method cannot be changed—because, a change will make the process useless.

(A) A review of the published research-work shows solid-evidence that the instant invention is ahead of its time looking at the present knowledge of the science.

At this stage science is trying to find a way to implant a new ‘light switch’ in the brain (see REF.-4) for treating mental-disorder(s), and/or to control sleep-wake state of the brain (see REF.-3). Science is unable to locate and ‘repair’ the ‘light switch’ which exists in the brain-stem, but stopped working. The process in the instant invention is able to ‘repair’ the ‘light switch’ that quit and make it work with no need for an implant, device or drug. The relevant articles discovered from a careful search of published material in the matter are cited, as REFs and copies attached.
REF.-1: Concept of the dominant hemisphere, p 719—Medical Physiology—Guyton & Hall, 11^{th} edition.
REF.-2: Sleepless Nights make grumpier Brains, October 2007.
REF.-3: Sleep-Wake controls identified, July 2007.
REF.-4: Beam of light—flips a switch in Brain, August 2007.
REF.-5: Counting sheep no aid to Insomnia, Oxford study.

(B) A summary of relevant findings from Oxford study, or the article retrieved by the Examiner known as CNN.com—‘Counting sheep no aid to Insomnia’ (REF.-5), is as follows:
The Oxford study is not identical to the instant invention as it (1) involves only one-thought:
   ‘imagine a scene’ or ‘count sheep’ or ‘none’; and (2) did not involve ‘visualization of 2 or more principal thoughts at the same time to split-mind’.
The Oxford study and the instant invention (see claim at 2.2) are in agreement on one small-part: the ‘selection of an object, location or action’ is critical to generate positive-signals in the mind—that study found—a thought to ‘imagine a scene’ works and a thought of ‘counting sheep’, the traditional cure of 19^{th} century, “does not work”.

The Oxford study confirms that ‘counting sheep’ does not work; whereas, the process in the instant invention has a chance to produce beneficial result, despite prior-Examiner's assertion the process is a ‘mental-exercise’ like ‘counting sheep’.

(C) The Examiner, now, asserts the method in the instant invention is identical to Oxford method (see Reply to office action @12, 16-18, below), knowing Oxford did not provide any method to use. Since the Oxford study did not describe or publish a ‘method’ that can produce a useful, tangible and concrete result as required under 35 USC @101, the Examiner cannot imagine to use that study as the basis under 35 USC @102 to review the instant invention.

More specifically, the Examiner concludes ‘as being anticipated by CNN.com’ (see office action mailed on Oct. 12, 2007); evidently, the natural-phenomena of streaming negative-thoughts into the mind did it—kill a novel process:

@12: “CNN discloses a method to induce sleep . . . activating the brain's master-clock to induce sleep by splitting the mind by concentrating, equally and calmly, upon two or more principal thoughts at the same time (counting sheep inherently requires concentrating in order to count, and two or more sheep (thoughts) which are counted . . . implies calmly thinking and that counting commands equal attention to the counted items)”

@16: “CNN discloses wherein the principal thoughts in step (b) are all visualizations of any objects, any locations, or any actions ( . . . a third group were left to their own devices implies any combination of the above . . . )”

@17: “CNN discloses where there are only two principal thoughts in step (b) (water fall or a beach . . . a third group were left to their own devices implies that . . . group could restrict their thoughts to two principals)”

@18: “CNN discloses wherein splitting the mind causes interference created havoc in motor-responses of user's brain that activates the brain's master-clock to induce sleep (splitting the mind by counting sheep . . . activates the brain's master-clock, in order to induce sleep as disclosed [in the instant invention])”
Reply—The above statements are factually incorrect. The article (1) did not report any such findings; and (2) did not report the use of 2 or more principal thoughts at the same time.

@14: “CNN discloses wherein a person using the method closes their eyes at the start and keeps them closed (counting sheep . . . )”.
Reply—The statement is incorrect. The article did not specify that.

@13: Examiner is able to imagine “instruction insomniacs received) to try different techniques is a training”.
Reply—If a simple statement like that is enough to train, why more is required in the instant invention. More is added in claim 2.0, setting forth the best mode contemplated by the inventor to carry out the invention under 35 USC @112.

@5: “the disclosure fails to address the variety and heterogeneity of the human race and their sleep habits . . . support was further not found . . . for the steps of the method for training a person . . . fail to indicate one skilled in the art how to train a person . . . without requiring undue experimentation, because of the vast differences in capability and willingness to be trained among the human race”; and

@8: “ . . . disclosing the type of person or environmental conditions surrounding the person . . . disclosing conditions of trainability of said person . . . ”
Reply—The above, and all other issues raised by the Examiner @2-9, and under 35 USC @112, @101 & @102, are addressed in claims 1.0, 2.0 and specification (also, continued below).

(D) A careful review of present scientific knowledge in the matter (see REFs 1 to 4) reveals a solid-proof in support of the instant invention that the method is ‘ahead’ of its time, as seen from the findings of the science below:
That brain has 2-hemispheres and one is the dominant;
That the 2-hemispheres communicate with each other, one thought at a time;
That any interference from 2 or more thoughts in the mind at the same time create havoc with mental-thoughts and motor-responses in the brain, and the 2-hemispheres stop communicating—a split mind (see REF.-1).
That split mind cause the light switch ‘flip’ in the brain-stem's reticular activating system (see REF.-3) to activate the master-clock and make brain fall-asleep or help cure depression or mental-disorder(s) etc. . . . (see REF.-2).
That the science is struggling to introduce a ‘protein or gene’ into the brain from outside to ‘fix’ depression and/or control sleep-wake state with a ‘flip’ of the light switch;
That the science is unable to locate and ‘repair’ the light switch which exists in the brain, but, stopped working . . . .
That the science is looking for a solution else-where (see REF.-3 & 4), but, not within where the solution lie.
That the instant invention looked for a solution within and found it, before the science can ever find a solution, as safe as the instant process!

(E) Now, turning to Examiner's other specific observations:

@7—“the invention as a whole encompasses a human being . . . claims are directed to the natural phenomena of thought . . . the invention is not a practical application of a natural phenomenon, because no device using the concepts is developed, and nothing more than the manipulation of basic ideas is described, and no article or physical object is transformed into a different state or thing”.
Reply—Yes, that's the purpose here to help benefit a human-being; Yes, claims are directed to cure a natural-phenomena of ‘intrusive-thoughts’ streaming into a sick-brain that stopped working; Yes, the invention is a “process” for practical application of a natural phenomenon based on solid concepts (see claims 1.0 & 2.0), and not a manipulation or a magic; Yes, brain is a physical object that gets “repaired and transformed” into a different state in the process as described; Yes, a “process” is patentable under 35 USC @101, see Parker 437 US 584, Funk Bro. Seed Co. 333 US 127, and Mackay Radio & Tel. Co. 306 US 86, where the court said—
   “ . . . when a claim containing a mathematical formula implements or applies that formula in a structure or process which when considered as a whole, is performing a function (transforming or reducing an article to a different state) which the patent laws were designed to protect . . . ”.

In the instant invention, the generation of positive-signals from principal thoughts is a mathematical formula; tuning in the new-signals to flip off the light switch in the brain is the application of the formula; and making the brain perform a function upon repair-transformation into a different state is a process as a whole which the patent laws must protect.

@8—“the claimed result cannot be assured, is unrepeatable, or unpredictable”.
Reply—An imaginative challenge—Yes, a quick demonstration of the method will erase that false image and show a clear picture that the claimed result is assured and repeatable to a person who learned the skill of the art.

@9—“claims merely evaluate abstract functions and do not bring about a substantial application . . . splitting the mind, do not cause an output or other indication to the user that something has occurred, because there is no physical result or output for a person using the method to confirm (the result) . . . (it) is not whether the steps taken to achieve a particular result are useful, tangible, or concrete, but whether the final result achieved by the claimed invention is useful, tangible and concrete”; and, the prior-Examiner(s) asserted that “mental-exercise is not patentable”.
Reply—The “process” involves (1) generating positive-signal data in the mind using principal thoughts, like writing a code-data on a piece of paper using thoughts; (2) inputting that signal data in the mind into the brain-algorithm, like inputting data from a piece of paper into a machine-algorithm in the computer; (3) tuning in the signal data in the brain-algorithm, like processing data in the machine-algorithm or computer-code; and (4) repairing and transforming brain-algorithm or the neurons to function and provide the desired output, a sleep-cycle, like repairing and transforming the machine-algorithm to function and provide a desired output. That's how the ‘process’ in Microsoft's machine-algorithm came into existence to function: Microsoft ‘repaired and transformed’ the ‘old-IBM’ machine-algorithm or process-code to make it work using new-data signals generated by a thought ‘process’, like in the instant invention. If the thought-process in Microsoft is Patentable, then, the process in the instant invention too is Patentable.

The invention is not a game of ‘abstract functions’ or a ‘mental-exercise’ for fun. It is a “process” to cure mental-disorder(s) with no adverse affect, unlike the drug or device. Thought is not an abstract function. It is the key to everything a human being can do. A set of thoughts can generate code-data to be written; a related set of thoughts can input that code-data to be processed; and, yet another related set of thoughts can make the code-data work to produce a desired output, however or where-ever one chooses. Similarly, a set of thoughts in this instant invention generate data in the mind, input that data into a non-functioning code in the brain, and keep processing the data until the code accepts it to become functional and able to produce a desired output, sleep. The ‘repair and transformation’ of brain to produce a desired output is not a natural phenomenon as it can only be achieved using the process described in the instant invention which is meant to benefit the human-being.

Yes, an abstract idea or even a so-called mental-exercise (thoughts) is patentable under 35 USC @101 & 102, if it can create a ‘novel and useful structure or process’ for a useful end. See Parker 437 US 584, Funk Bro. Seed Co. 333 US 127, and Mackay Radio & Tel. Co. 306 US 86, where the court said—

“A process is not unpatentable simply because it contains a law of nature or algorithm (like in the instant invention—thoughts in mind or algorithm in brain) . . . if there is to be an invention (even from an abstract idea), it must come from the application of the law to a new and useful end . . . while a scientific truth is not a patentable invention, a novel and useful structure or process created with the aid of knowledge of scientific truth may be . . . (when the process performs a function)”.

On the issue of “splitting the mind, do not cause an output or other indication to the user that something has occurred, because there is no physical result or output for a person using the method to confirm (the result) . . . ”—

Yes, that's true in many other instances too: for example, in an electric-shock or radiation or oxygen therapy, a process (Patented and/or Approved) when used the person cannot see the input signals, the process in ‘repair and transformation’ of cells or the output of a different physical state. Although the person may never know of any physical output, being alive he/she knows the process has benefited. In such cases, like here, a Physician/Technician can monitor, read and record the output of signal-activity in the cells or brain when awake with streaming intrusive thoughts and when asleep using the process.

The output confirms the result; and the person will know of the beneficial result upon awakening from a restful sleep. The same can be said of sleeping-pills, except the user gets up with a tired-drowsy feeling or headache. The process and the output can be demonstrated, if required.

On the issue “(it) is not whether the steps taken to achieve a particular result are useful, tangible, or concrete, but whether the final result achieved by the claimed invention is useful, tangible and concrete”—

Yes, that's true. The claims 1.0 & 2.0 clearly stated that the final result achieved by the method is useful, tangible and concrete, and the result is repeatable. So, if the intent (of the “in house authorities”—see interview of May 30, 2007 with Dr.Pezzuto) is not to favor drugs-devices, and not discriminate against the process in the instant invention or millions of People that can benefit, the Examiner should accept inventor's claims or allow a quick demonstration of the method to show the benefit is real, repeatable for practical application, and the final result produced is useful, tangible and concrete.

@19—Prior arguments:

(a) U.S. Pat. No. 5,318,503, issued on Jun. 7, 1994, to Robert F. Lord, discloses a method and apparatus for auditory and olfactory relaxation, using a headset that both generates sound and diffuses fragrance. The instant invention is distinguishable, in that it requires no apparatus, and it induces sleep rather than merely relaxation.

(b) U.S. Pat. No. 5,599,274, issued on Feb. 4, 1997, to Nusa Widjaja and Robert W. Fish, discloses a trophotropic response system, which uses both light and sound to relax the user. Again, the instant invention is distinguishable, in that it requires no apparatus, and it induces sleep rather than merely relaxation.

(c) U.S. Pat. No. 6,641,522, issued on Nov. 4, 2003, to Joseph August, discloses an apparatus for projecting “biophilic” natural landscape scenes.

(d) U.S. Pat. No. 6,641,523, issued on Nov. 4, 2003, to Matthew Ashenden, discloses an apparatus for reducing stress that combines music and words.

None of the above inventions and patents, taken either singly or in combination, is seen to describe the instant invention as claimed to induce sleep.

### SUMMARY OF THE INVENTION

The present invention in its first preferred embodiment is a method to induce sleep, and include 2-steps:

1. Calm mind of intrusive thoughts to prepare for a sleep cycle by calmly visualizing any one principal thought (which can be any object-thing, location-spot or action-item etc. . . . ).

2. Split mind to activate the master clock and induce a sleep cycle by calmly visualizing any two or more principal thoughts at the same time (which can be any, see at 1, above).

The present invention in its second preferred embodiment is a method to teach the first embodiment.

Accordingly, it is the principal object of the invention to calm mind and split mind to induce sleep to a person who otherwise is unable to go to sleep naturally.

It is an object of the invention to alleviate stress, anxiety, anger, mental-distress, depression, neurodegenerative and neurological disorders; and to alleviate health problems due to high blood pressure, hypertension, hot flashes, obesity and cancer.

It is an object of the invention to provide a method that any person, irrespective of culture, religion, nationality, sex or rich-poor could be able to use and derive benefit in an inexpensive, dependable and effective way and without drugs and devices.

These and other objects of the present invention will become readily apparent upon further review of the following specification, drawings and references.

### BRIEF DESCRIPTION OF THE DRAWINGS

FIG. 1, a flowchart, shows the process steps performed in the first preferred embodiment of the invention, and taught in the second preferred embodiment of the invention.

FIG. 2, picture, shows a rear view of 2-hemispheres of the Brain.

FIG. 3.1, sketch, shows a sectional view of right-hemisphere of the Brain looking from inside: (1) it identifies the location of sustantio nigra which produces dopamine (melatonin), and the pathways of melatonin into thalamus where it decreases the signal-activity that goes into the cortex; and (2) it identifies the location of brain-stem which turns-off the light switch (when split-mind occurs) allowing serotonin to take pathways into thalamus-hypothalamus & cortex to activate the master-clock and begin the sleep cycle.

FIG. 3.2, sketch, shows a sectional view of left-hemisphere of the Brain looking from inside: it identifies the locations for excitatory and inhibitory areas in the brain-stem: the upper brain-stem sends signals into thalamus to get them excited; then, thalamus sends those excited signals into appropriate regions of the brain-cortex; whereas, the lower brain-stem cuts-off the signals going out of upper brain-stem (when split mind occurs) and flips off the light switch.

FIG. 3.3, sketch, shows the key parts in the Brain where dopamine (melatonin) and serotonin are made.

### DETAILED DESCRIPTION OF THE PREFERRED EMBODIMENTS

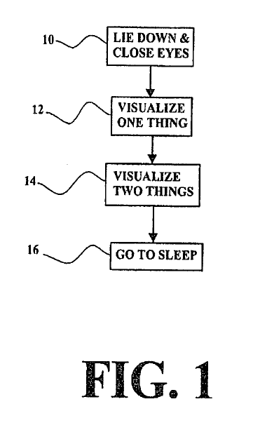

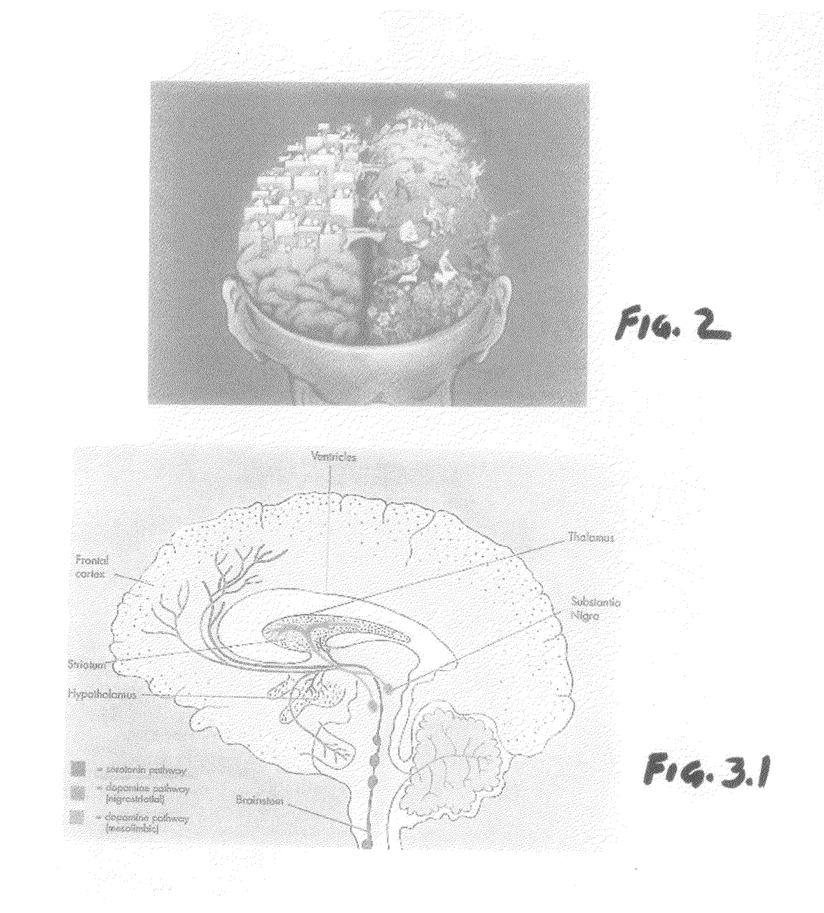

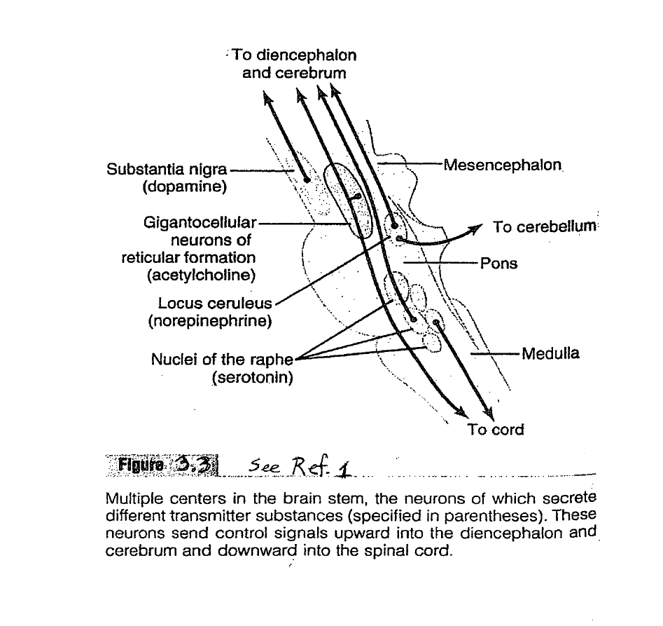

The instant invention in its first embodiment is a method to induce sleep, as shown in the steps in FIG. 1. First, the person using the method will sit/lay in a sofa/bed and closes his/her eyes 10. The person then calmly concentrates on one principal thought (visualizing an object-thing, location-spot or action-item etc.) to calm mind of intrusive thoughts streaming into the mind, as a natural phenomena, due to anxiety 12. The decrease of excited-signal activity in the cortex cause the pineal gland make and release melatonin into the bloodstream inviting sleep and calms the brain-stem and thalamus-hypothalamus (see FIGS. 3.2, 3.3 & 3.1).

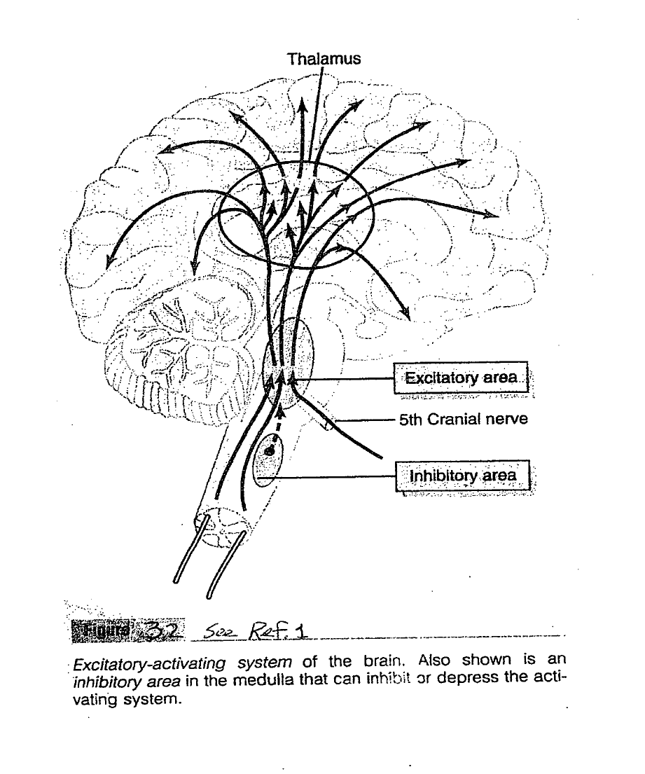

The person next calmly concentrates on two or more principal thoughts simultaneously, equally and calmly (visualizing objects-things, locations-spots, actions-items, or any combination) 14, while keeping his/her eyes closed, until the cerebrum (see FIG. 2) can no longer communicate between its left and right hemispheres (using the corpus callosum and anterior commissure). Because the brain normally processes only one conscious principal thought at a time, simultaneously processing more than one conscious thought interferes with communication between the two hemispheres of the brain (see REF.-1). That is a split mind with the non-dominant side of the brain turned-off (which is the right hemisphere in right-handed person) and not participating in interpreting visual patterns due to interference created havoc in motor responses of the cerebrum. That will cause the lower brain-stem's reticular inhibitory area (located between pons-medulla) to inhibit the upper brain-stem's reticular excitatory area (see FIGS. 3.2 & 3.3), and the activity in the cortex decreases instantaneously. The acetylcholine system of reticular formation in brain-stem secretes neurotransmitter hormonal agents into the substance of the brain to provide long-period control of activity in specific brain regions. The dopamine system of substantia nigra (located near mesencephalon) act as an inhibitory transmitter in basalganglia and send signals to neurons in the caudate nucleus and putamen located in the cerebrum, and secrete dopamine to thalamus-hypothalamus and limbic system in the brain-stem (see FIGS. 3.1 & 3.3).

Thus, the activity in cortex, thalamus-hypothalamus and brain-stem gets silenced in preparation for a sleep-state brain. The serotonin system of raphe nuclei (located in brain-stem, see FIG. 3.3) secrete serotonin, while melatonin is secreted by the pineal gland, and both cause sleep in specific brain regions, activating brain's master-clock, a special center called the supra-chiasmatic nucleus (SCN) in thalamus-hypothalamus, and begins the sleep-cycle. The instant invention is a process in which brain-neurons transform into a different-physical state and produce melatonin and serotonin without the use of any drug or device to be imported from outside. The REM sleep is probably caused by norepinephrine located in brain-stem between pons-mesencephalon (see FIG. 3.3), which generally excites the Brain and at the same time inhibits a few Brain regions or areas. Thus, the person goes to sleep 16.

The instant invention in its second embodiment is a method to teach a person for sleep, as illustrated in the steps in FIG. 1. This preferred embodiment of the invention comprises teaching a person to practice-learn the first preferred embodiment.

The invention is not a natural phenomenon in falling asleep, because an untrained person, who cannot fall-asleep naturally, is not likely to be aware of the process or the skill of the art to use on his/her own to achieve benefit from the method. Using the method to induce sleep by tuning-in positive-signals to create havoc in brain's motor responses and cause split mind is not a natural process. The person who cannot fall-asleep without the use of drugs must have lost his/her brain's natural ability to fall-asleep; any process or drug or device that can transform such a brain to restore its ability to fall-asleep, must be an invention. A person, whose brain's natural ability to make melatonin and serotonin has failed will be able to repair and transform his/her brain to make melatonin and serotonin and induce sleep using the method described in the instant invention, without drug or device.

It is to be understood that the instant invention is not limited to the embodiments described above, but encompasses any and all embodiments within the scope of the claims.

1. Prasanti-loka (Bliss in mind):
1.0 The process in the method described will create bliss in mind and induce sleep to the brain, a sleep-cycle of deep-sleep (DS) and rapid-eye-movement sleep (REM). The process generates positive signals in the mind to tune the brain for the benefit of a person who is unable to fall-asleep because of the excited signals being generated from natural phenomena of streaming intrusive thoughts into the mind due to worry, anxiety, anger, depression or mental-disorder(s).
1.1 The process ‘repairs and transforms’ the brain into a different-physical state to restore its ability to produce melatonin (dopamine) and serotonin, and activate the master clock in the brain for a sleep-cycle by generating positive-signals in the mind, inputting positive-signals into the process-algorithm in the brain, tuning-in the positive-signals to flip off the ‘light-switch’ in the brain-stem and cut off the excited-signals into the brain, and producing hormones to activate the master-clock in the brain and begin a sleep-cycle as the output.
1.2 The 2-step process in the method tunes the mind by (1) decreasing the frequency of the excited-signals in the brain-cortex, and (2) cutting off the excited-signal activity across the neurons in the reticular activating system of the brain-stem, as follows:
(A) Initiate tuning by concentrating upon one principal thought: To create a calm mind by decreasing the frequency of excited-signals in the brain-cortex, the person is required to concentrate upon one principal thought in the mind. The process (1) generates 1-set of positive-signals in the mind; (2) inputs that data into the process-algorithm in the brain; (3) tunes the new-signals to replace the old-signals in the brain-cortex; and (4) decreases the frequency of excited-signals in the cortex to cause calm mind and the brain produce melatonin, a sleep inviting hormone.
(B) Continue the tuning by concentrating upon two or more principal thoughts: To create bliss in mind and induce sleep by cutting off all excited-signals into the brain, the person is required to concentrate equally and calmly upon two or more principal thoughts in the mind at the same time. The process (1) generates 2-sets of positive-signals in the mind; (2) inputs that data into the process-algorithm in the brain; (3) tunes the 2-sets of new-signals, similar to tuning the ‘out-of-phase’ waves to cancel each other, to cause an ‘interference created havoc in the brain's motor-responses’ and split the mind to stop the 2-hemispheres of the brain from communicating with each other; and (4) causes the ‘light switch’ in the brain-stem to flip off and cut off all excited-signals coming into the brain to make the brain produce serotonin and activate the master-clock to put the brain in a sleep-cycle. The brain that was unable to sleep before begins to function having been ‘repaired and transformed’ into a different-physical state in the process.
1.3 The instant invention is ‘ahead’ of its time as the present knowledge of science is looking to implant a new ‘light switch’ in the brain to flip on-off, and treat mental-disorder(s) and/or control the sleep-wake state of the brain; science is unable to locate and ‘repair’ the ‘light switch’ which already exists in the brain-stem. The instant invention is able to ‘repair’ that ‘light switch’ which stopped working.
1.4 In the method to induce sleep in claim 1.0, a person is required to sit/lay in a sofa/bed and close his/her eyes at the start of step 1.2(A) and keep them closed through step 1.2(B) to cut off incoming light into the eyes and get dark-night's sleep.
1.5 In the method to induce sleep in claim 1.0, a person is required to generate positive-signals in the mind and input that new data into the process-algorithm at step 1.2(A) by visualizing one principal thought about any object-thing, location-spot, or action-item that exist within or without.
1.6 In the method to induce sleep in claim 1.0, a person is required to generate positive-signals in the mind and input that new data into the process-algorithm at step 1.2(B) by visualizing two or more principal thoughts about any objects-things, locations-spots, actions-items, or combinations that exist within or without.
1.7 The method to induce sleep in claim 1.0, will produce a useful, tangible, concrete result for any person of any variety or heterogeneity of race with different sleeping habits, who has learned the skill of the art or the method described at 1.2(A) and (B) above.
(a) The positive-signals generated in the process to ‘repair and transform’ brain-neurons and produce a desired output is not a natural phenomenon as it can only be achieved using the method described in the instant invention.
(b) The invention helps create bliss in mind and benefits any human-being who is unable to be free of intrusive thoughts streaming into the mind as natural phenomena. Given the calming positive-signals as the new data input into the process-algorithm in the brain the excited-signals from streaming intrusive thoughts are replaced, and the ‘repair and transformation’ of the brain-neurons into a different-physical state induces sleep as the new output.
(c) The beneficial result can be demonstrated and confirmed by the change in the recorded output of brain-activity when awake and while asleep.
(d) The person using the method can sense the benefit or physical result after awakening from restful sleep. The same can be said of the user of sleeping-pills, except, that person gets up with a tired-drowsy feeling or headache.

2. Method to Teach:
2.0 The best mode to carry out the invention is for a person (of any variety or heterogeneity of race with vast difference in sleeping habits) willing to learn the art to follow the steps, below:
(A) The person willing to learn is required to sit/lay in a sofa/bed with closed-eyes, and practice to visualize upon one principal thought, using one of those selected below at 2.2, until calm mind is attained with decrease of intrusive thoughts in the mind. Such practice should continue with different principal thoughts, one at a time, until ‘one-thought’ which works consistently is identified.
(B) The person willing to learn is required to continue step (A), and practice to visualize upon two or more principal thoughts at the same time, equally and calmly, using one-set of those selected below at 2.2, until a progressive tuning-in of ‘signal-interference creates havoc in brain's motor-responses’ and splits the mind to make the brain fall-asleep. Such practice should continue with different principal thoughts, two or more at a time, until ‘one-set’ of thoughts which works consistently is identified.
2.1 Any person who wants to be an ‘ordinary artisan skilled in the art’ or wants to learn the use of the method for his/her benefit should get proper training to acquire the skill because the method is not a natural-process like the natural-phenomenon of intrusive thoughts streaming into an insomniac brain due to anxiety, depression etc.
(a) Work is in progress to develop a ‘device’ that can assist a person to learn and practice the method in achieving the desired result.
(b) The method has no environmental and/or learning conditions provided the person is willing to learn.
2.2 A person willing to learn must identify the nature of intrusive thoughts in order to select appropriate principal-thoughts for practice in generating positive-signals.
(a) Such a finding can help select a set of objects-things, locations-spots, actions-items or combinations to use as principal thoughts in the practice of the method.
2.3 In the method to induce sleep in claim 2.0, a person is required to learn to sit/lay in a sofa/bed and close his/her eyes at the start of step (A) and keep them closed through step (B) to cut-off incoming light into the eyes and get dark-night's sleep.
2.4 In the method to induce sleep in claim 2.0, a person is required to learn to generate positive-signals in the mind and input that new data into the process-algorithm at step (A) by visualizing one principal thought about any object-thing, location-spot or action-item that exist within or without.
2.5 In the method to induce sleep in claim 2.0, a person is required to learn to generate positive-signals in the mind and input that new data into the process-algorithm at step (B) by visualizing two or more principal thoughts about any objects-things, locations-spots, actions-items, or combinations that exist within or without.
2.6 Any person who learned the method to induce sleep in claim 2.0, can produce a useful, tangible, concrete result whenever that person utilizes the process to (1) decrease intrusive thoughts in the mind, and (2) tune-in the signal-interference created havoc in brain's motor-responses to split the mind. The split mind transforms brain-neurons into a different-physical state to flip off the ‘light switch’ in the brain-stem. The brain having ‘repaired and transformed’ produces melatonin to invite sleep and serotonin to activate the master-clock in the brain and begin a sleep-cycle.
2.7 The result achieved in the instant invention is repeatable for practical application, and the final result produced is useful, tangible and concrete.
